(19) United States
(12) Patent Application Publication
(10) Pub. No.: US 2024/0130790 A1
(43) Pub. Date: Apr. 25, 2024

(54) DYNAMIC TISSUE IMAGERY UPDATING

(71) Applicant: KONINKLIJKE PHILIPS N.V., EINDHOVEN (NL)

(72) Inventors: Torre Michelle BYDLON, MELROSE, MA (US); Sean Joseph KYNE, BROOKLINE, MA (US); Paul THIENPHRAPA, CAMBRIDGE, MA (US)

(21) Appl. No.: 17/768,262

(22) PCT Filed: Oct. 15, 2020
(86) PCT No.: PCT/EP2020/079273

§ 371 Date: Apr. 11, 2022

Related U.S. Application Data
(60) Provisional application No. 62/916,348, filed on Oct. 17, 2019.

(51) Int. Cl. A61B 34/10; A61B 34/00; A61B 34/20; G06T 1/00; G06T 7/00; G06T 7/20; G16H 30/40

(52) U.S. Cl. CPC A61B 34/10; A61B 34/25; G06T 1/0007; G06T 7/0016; G06T 7/20; G16H 30/40; A61B 2034/105; A61B 2034/2048

(57) ABSTRACT

A controller (122) includes a memory (12220) that stores instructions and a processor (12210) that executes the instructions. When executed, the instructions cause the controller (122) to implement a process that includes obtaining (S405) pre-operative imagery of the tissue in a first modality, registering (S425) the pre-operative imagery of the tissue in the first modality with a set of sensors (195-199) adhered to the tissue, and receiving (S435), from the set of sensors (195-199), sets of electronic signals for positions of the set of sensors (195-199). The process also includes computing (S440) geometry of the positions of the set of sensors (195-199) for each set of the sets of electronic signals and computing (S450) movement of the set of sensors (195-199) based on changes in the geometry of the positions of the set of sensors (195-199) between sets of electronic signals from the set of sensors (195-199). The pre-operative imagery is updated to reflect changes in the tissue based on movement of the set of sensors (195-199).

### BACKGROUND

An interventional medical procedure is an invasive procedure to the body of a patient. Surgery is an example of an interventional medical procedure and is the treatment of choice for a number of ailments including for some forms of cancer. In cancer surgery, an organ that includes the cancerous tissue (tumor) is often soft, flexible, and easily manipulated. Pre-operative imagery of the organ that includes the cancerous tissue is used to plan the surgical resection (removal) of the cancerous tissue in cancer surgery. For example, medical clinicians, such as surgeons, may identify the location of the cancerous tissue on the organ in pre-operative imagery and mentally plan a path to the cancerous tissue based on the pre-operative imagery. During surgery, the clinician begins following the planned path to the cancerous tissue by manipulating anatomy such as by pushing the organ, pulling the organ, cutting the organ, cauterizing the organ, and dissecting the organ. When the organ that includes the cancerous tissue is very soft, these manipulations cause the organ to distort and therefore the anatomy of the organ is different compared to the pre-operative imagery of the organ.

Additionally, some organs such as brains and lungs will drastically shift or change shape due to a pressure change when a hole is cut into the body. A brain shift occurs when a hole is created in the skull. In lung surgery, the lung collapses when a hole is created in the chest cavity. Thus, three-dimensional (3D) anatomy that includes cancerous tissue can change due to pressure differentials or manipulations of the anatomy.

Changes in the three-dimensional anatomy that includes cancerous tissue can be confusing to the clinician and, in practice, the clinician may be forced to reorient their perspective relative to the pre-operative imagery and the initial surgical plan. To reorient, clinicians may have to move, stretch, flip, and rotate the anatomy to identify known landmarks, and these additional manipulations may further change the anatomy compared to pre-operative imagery and therefore sometimes add to the overall disorientation. Dynamic tissue imagery updating described herein addresses these challenges.

### SUMMARY

According to an aspect of the present disclosure, a controller for dynamically updating imagery of tissue during an interventional medical procedure includes a memory that stores instructions and a processor that executes the instructions. When executed by the processor, the instructions cause the controller to implement a process that includes obtaining pre-operative imagery of the tissue in a first modality and registering the pre-operative imagery of the tissue in the first modality with a set of sensors adhered to the tissue for the interventional medical procedure. The process implemented when the processor executes the instructions also includes receiving, from the set of sensors, sets of electronic signals for positions of the set of sensors, and computing geometry of the positions of the set of sensors for each set of the sets of electronic signals. The process implemented when the processor executes the instructions further includes computing movement of the set of sensors based on changes in the geometry of the positions of the set of sensors between sets of electronic signals from the set of sensors and updating the pre-operative imagery to updated imagery to reflect changes in the tissue based on the movement of the set of sensors.

According to another aspect of the present disclosure, an apparatus configured to dynamically update imagery of tissue during an interventional medical procedure includes a memory that stores instructions and pre-operative imagery of the tissue obtained in a first modality. The apparatus also includes a processor that executes the instructions to register the pre-operative imagery of the tissue in the first modality with a set of sensors adhered to the tissue for the interventional medical procedure. The apparatus further includes an input interface via which sets of electronic signals are received, from the set of sensors, for positions of the set of sensors. The processor is configured to compute geometry of the positions of the set of sensors for each set of the sets of electronic signals and to compute movement of the set of sensors based on changes in the geometry of the positions of the set of sensors between sets of electronic signals from the set of sensors. The apparatus updates the pre-operative imagery to updated imagery that reflects changes in the tissue based on the movement of the set of sensors and controls a display to display the updated imagery for each set of electronic signals from the set of sensors.

According to yet another aspect of the present disclosure, a system for dynamically updating imagery of tissue during an interventional medical procedure includes a sensor and a controller. The sensor is adhered to the tissue and includes a power source that powers the sensor, an inertial electronic component that senses and processes the movement of the sensor, and a transmitter that transmits electronic signals indicating the movement of the sensor. The controller includes a memory that stores instructions and a processor that executes the instructions. When executed by the processor, the controller implements a process that includes obtaining pre-operative imagery of the tissue in a first modality and registering the pre-operative imagery of the tissue in the first modality with the sensor. The process implemented when the processor executes the instructions also includes receiving, from the sensor, electronic signals for movement sensed by the sensor and computing geometry of the sensor based on the electronic signals. The process implemented when the processor executes the instructions further includes updating the pre-operative imagery to reflect changes of the tissue based on the geometry.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A is a simplified schematic block diagram of a system for dynamic tissue imagery updating, in accordance with a representative embodiment.

FIG. 1B illustrates a controller for dynamic tissue imagery updating, in accordance with a representative embodiment.

FIG. 1C illustrates an operational progression for sensors in dynamic tissue imagery updating, in accordance with a representative embodiment.

FIG. 1D illustrates a method for dynamic tissue imagery updating for the operational progression for sensors in FIG. 1C, in accordance with a representative embodiment.

FIG. 2A illustrates another method for dynamic tissue imagery updating, in accordance with a representative embodiment.

FIG. 2B illustrates sensor movement for the method for dynamic tissue imagery updating in FIG. 2A, in accordance with a representative embodiment.

FIG. 3 illustrates a sensor for dynamic tissue imagery updating, in accordance with a representative embodiment.

FIG. 4 illustrates another method for dynamic tissue imagery updating, in accordance with a representative embodiment.

FIG. 5 illustrates another operational progression for sensors in dynamic tissue imagery updating, in accordance with a representative embodiment.

FIG. 6 illustrates an arrangement of sensors on tissue in dynamic tissue imagery updating, in accordance with a representative embodiment.

FIG. 7 illustrates another method for dynamic tissue imagery updating, in accordance with a representative embodiment.

FIG. 8 illustrates a sensor placement in dynamic tissue imagery updating, in accordance with a representative embodiment.

FIG. 9 illustrates another operational progression for sensors in dynamic tissue imagery updating, in accordance with a representative embodiment.

FIG. 10 illustrates a user interface for an apparatus monitoring sensors in dynamic tissue imagery updating, in accordance with a representative embodiment.

FIG. 11 illustrates a general computer system, on which a method for dynamic tissue imagery updating may be implemented, in accordance with another representative embodiment.

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. The terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the present disclosure.

As used in the specification and appended claims, the singular forms of terms “a”, “an” and “the” are intended to include both singular and plural forms, unless the context clearly dictates otherwise. Additionally, the terms “comprises”, and/or “comprising,:” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims.

As described herein, deformations of tissue due to, for example, pressure differentials or manipulation of the tissue may be tracked and the tracking may be used to update pre-operative imagery of the tissue so as to be aligned with the surgical state of the tissue. The deformation of the tissue may be tracked using sensors configured to provide positional and/or movement related data of the sensors and corresponding locations of the tissue. The tracking of positions and/or movement of the sensors may be used to morph the pre-operative imagery of the tissue into updated imagery of the tissue. The updated imagery of the tissue may be used by clinicians to better visualize anatomy during the interventional medical procedure. The anatomy as seen through updated imagery that better matches the actual surgical state may result in improved treatment.

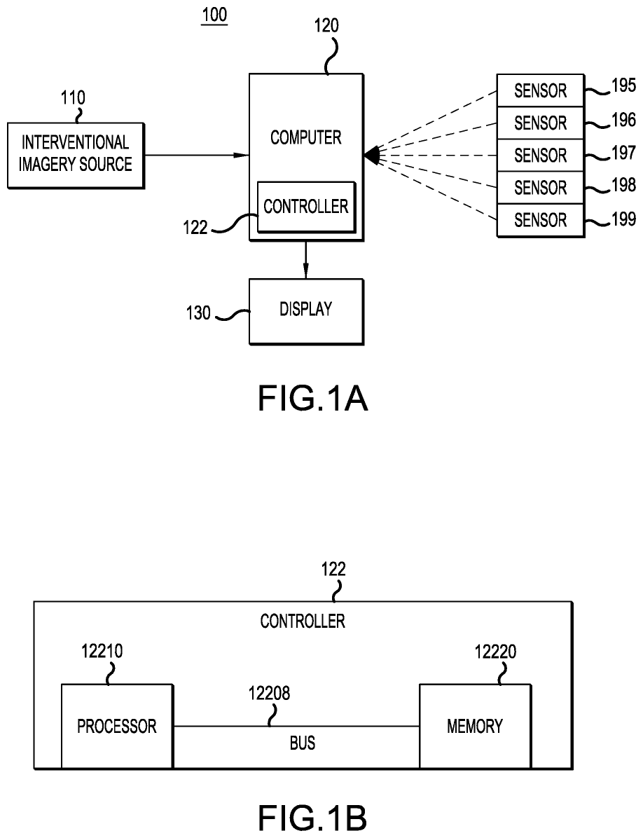

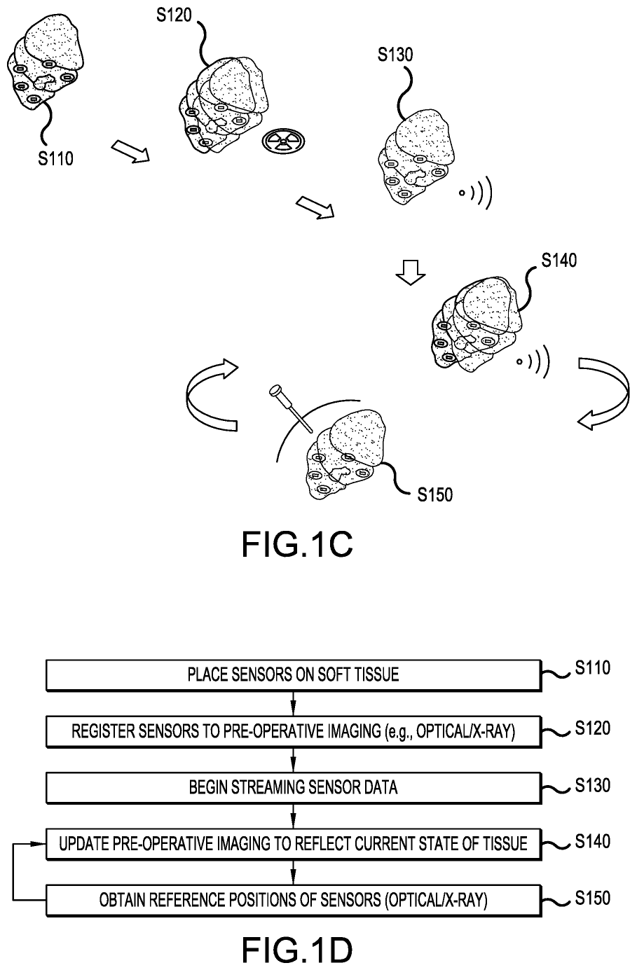

FIG. 1A is a simplified schematic block diagram of a system 100 for dynamic tissue imagery updating, in accordance with a representative embodiment.

As shown in FIG. 1A, the system 100 includes an interventional imagery source 110, a computer 120, a display 130, a first sensor 195, a second sensor 196, a third sensor 197, a fourth sensor 198 and a fifth sensor 199. The system 100 may include some or all components of a dynamic tissue imagery updating system described herein. The system 100 may implement some or all aspects of the methods and processes of the representative embodiments described below in connection with FIGS. 1C, 1D, 2A, 4 and 7.

The interventional imagery source 110 may be an endoscope such as a thoracoscope, for example, elongated in shape and used within the thoracic cavity (which includes the heart) for examination, biopsy and/or resection (removal) of diseased tissue. Other types of endoscopes may be incorporated without departing from the scope of the present teachings. The interventional imagery source 110 may also be CT system, a CBCT system, an X-Ray system, or another alternative to an endoscope such as a thoracoscope. The interventional imagery source 110 may be used in video assisted thoracic surgery (VATS) within the pleural cavity (which includes the lungs) and the thoracic cavity. The interventional imagery source 110 sends interventional imagery such as endoscopic video to the computer 120 via a wired connection and/or via a wireless connection such as BLUETOOTH® or 5G, for example. The interventional imagery source 110 may be used to image the tissue of the organ subject to surgery, though the pre-operative imagery described herein may exist independent of the interventional imagery source 110 such as when the pre-operative imagery is obtained by CT imaging and the interventional imagery source 110 is an endoscope.

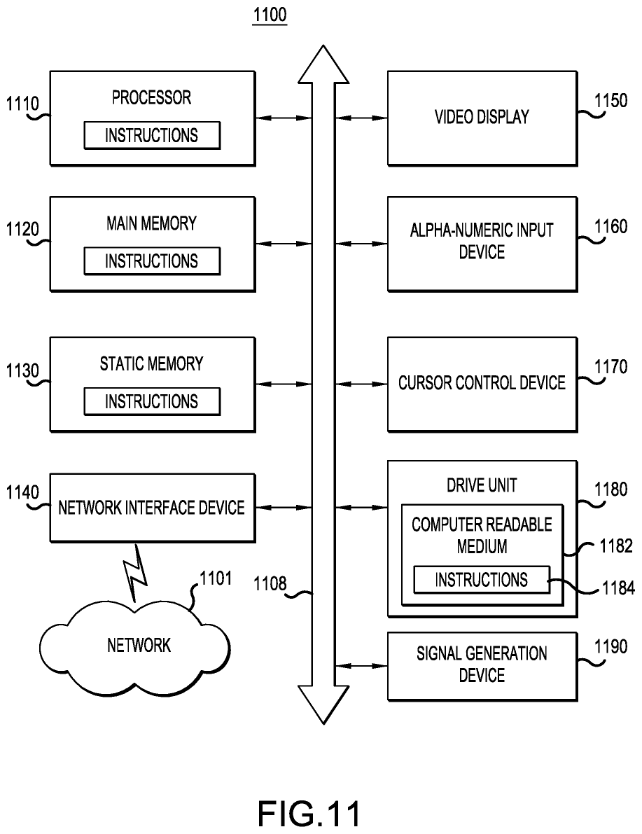

The computer 120 includes at least the controller 122 but may include any or all elements of an electronic device such as in the computer system 1100 of FIG. 11, explained below. For example, the computer 120 may include ports or other types of communications interfaces to interface with the interventional imagery source 110 and the display 130. The controller 122 includes at least a memory that stores software instructions and a processor that executes the software instructions to directly or indirectly implement some or all aspects of the various processes described herein. The computer 120 may include some or all components of a dynamic tissue imagery updating computer described herein. The computer 120 may implement some or all aspects of the methods and processes of the representative embodiments described below in connection with FIGS. 1C, 1D, 2A, 4 and 7.

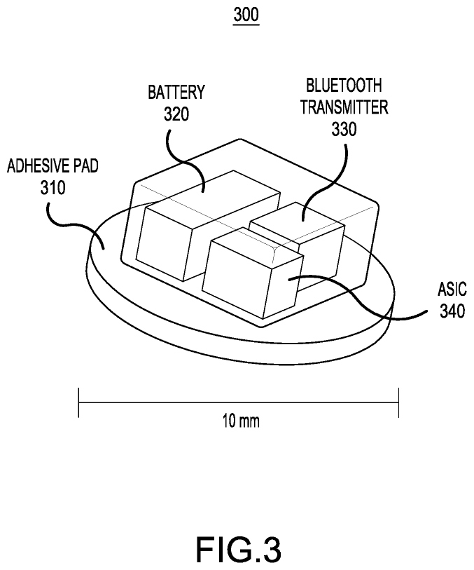

The computer 120 may be configured to communicate with the first sensor 195, the second sensor 196, the third sensor 197, the fourth sensor 198 and the fifth sensor 199 using a wireless protocol such as BLUETOOTH®, or by another suitable communication protocol. The first sensor 195 to the fifth sensor 199 are attached to an organ (e.g., lung) to be imaged. Although five sensors are shown in some embodiments herein, dynamic tissue imagery updating is not limited to five sensors. For example, a set of sensors may include as few as one sensor, and as many as five or more sensors. A representative example of a single sensor is shown in and described with respect to FIG. 3 below and includes a transmitter 330. Accordingly, the computer 120 may include a BLUETOOTH® interface such as a transceiver or may have such a BLUETOOTH® interface connected thereto. For example, a BLUETOOTH® interface may be plugged into a port on the computer 120. The computer 120 may also be connected to the display 130 via a wire plugged into another port or other type of interface. The computer 120 may also be connected to the display 130 and other equipment such as cameras by additional ports or other types of interfaces.

The controller 122 may include a combination of a memory that stores software instructions and a processor that executes the instructions. The controller 122 may be implemented as a stand-alone component with the memory and processor as in FIG. 1B described below, outside of a computer 120 or a system 100. Additionally, a controller 122 may be implemented in or with other devices and systems, including in or with a smart monitor, or in or with a dedicated medical system such as a system for medical imaging including an MRI system or an X-ray system. The controller 122 may implement some or all aspects of the methods and processes of the representative embodiments described below in connection with FIGS. 1C, 1D, 2A, 4 and 7, by executing software. Updates to the pre-operative imagery may be performed by the controller 122 of the computer 120 based on data from the five sensors including the first sensor 195, the second sensor 196, the third sensor 197, the fourth sensor 198 and the fifth sensor 199.

For example, the controller 122 may obtain pre-operative imagery of tissue via a memory stick or drive plugged into the computer 120, or via an internet connection in or on the computer 120. The controller 122 may receive sets of electronic signals from the first sensor 195, the second sensor 196, the third sensor 197, the fourth sensor 198 and the fifth sensor 199 via the BLUETOOTH® connection and register the pre-operative imagery of the tissue to the sensors. The registration by the controller 122 may be based on initial sets of electronic signals from the sensor(s) on an organ. The controller 122 may thereafter update the pre-operative imagery based on changed positions of the sensors as reflected in the subsequent sets of electronic signals. The controller 122 may superimpose the pre-operative imagery as updated on interventional imagery such as endoscopic imagery from the interventional imagery source 110 on the display 130. Alternatively, the controller 122 may generate two separate image displays for the pre-operative imagery and the interventional imagery from the interventional imagery source 110.

The display 130 may be a video display that displays endoscopic imagery or other interventional imagery derived from the interventional imagery source 110 and/or any other imaging equipment present in the environment where the interventional medical procedure takes place. The display 130 may be a monitor or television that displays video in color or in black and white. The display 130 may be a specialized interface for displaying endoscopic imagery, or another type of electronic interface that displays video such as endoscopic imagery of tissue from the pre-operative state through a series of updates. The display 130 may include touch-screen functionality to accept input directly from an operator. The display 130 also displays the pre-operative imagery and the updated imagery based on the pre-operative imagery, such as by superimposing the pre-operative imagery over the endoscopic imagery in a section. Alternatively, the display 130 may display the endoscopic imagery and the pre-operative imagery/updated imagery side-by-side. In another embodiment, the display 130 includes two or more separate physical displays connected to the computer 120, and the endoscopic imagery and the pre-operative imagery/updated imagery are displayed on separate physical displays connected to the computer 120 and controlled by the controller 122.

The first sensor 195, the second sensor 196, the third sensor 197, the fourth sensor 198 and the fifth sensor 199 may be substantially identical in terms of physical and operational characteristics. The first sensor 195 to the fifth sensor 199 may each be provided with a unique identification to be transmitted each time sensor data is transmitted. The first sensor 195 to the fifth sensor 199 may also each include a gyroscope, an accelerometer, a compass, and/or any other component usable to localize the position of the sensor in a common three-dimensional coordinate system. The first sensor 195 to the fifth sensor 199 may also each include a microprocessor that executes instructions to generate the sensor data based on readings from the gyroscope, accelerometer, compass and/or other component. An embodiment of a sensor that is representative of the first sensor 195, the second sensor 196, the third sensor 197, the fourth sensor 198 and the fifth sensor 199 is shown in FIG. 3 and described below.

FIG. 1B illustrates a controller for dynamic tissue imagery updating, in accordance with a representative embodiment.

The controller 122 includes a memory 12220, a processor 12210, and a bus 12208 that connects the memory 12220 and the processor 12210. The controller 122 may include components for implementing some or all aspects of the methods and processes of the representative embodiments described below in connection with FIGS. 1C, 1D, 2A, 4 and 7. The controller 122 is shown as a stand-alone device in FIG. 1B insofar as a controller 122 is not necessarily a component in or connected to the computer 120 in FIG. 1A. For example, a controller 122 may be provided as a chipset, such as by a system on chip (SoC). However, the controller 122 may alternatively be connected to the computer 120 as a peripheral component such as an adapter plugged into a port on the computer 120. The controller 122 may also be implemented in or connected directly to other equipment such as the display 130 in FIG. 1A, a laptop computer, a desktop computer, a smartphone, a tablet computer, or medical equipment present during an interventional medical procedure described herein.

The processor 12210 is fully explained by the descriptions of a processor in the computer system 1100 of FIG. 11 below. The processor 12210 may execute software instructions to implement some or all aspects of the methods and processes of the representative embodiments described below in connection with FIGS. 1C, 1D, 2A, 4 and 7.

The memory 12220 is fully explained by the descriptions of a memory in the computer system 1100 of FIG. 11 below. The memory 12220 stores instructions and pre-operative imagery of the tissue obtained in a first modality. The memory 12220 stores the software instructions executed by the processor 12210 to implement some or all aspects of the methods and processes described herein.

The memory 12220 may also store pre-operative imagery of the tissue that is subject to dynamic tissue imagery updating. The pre-operative imagery of the tissue may be obtained in a first modality such as by MRI, CT, CBCT or X-ray imagery. Intraoperative imagery of the tissue may be obtained in a second modality such as via the interventional imagery source 110 in FIG. 1A. The bus 12208 connects the processor 12210 and the memory 12220.

The controller 122 may also include one or more interfaces (not shown) such as a feedback interface to send data back to the clinician. Additionally or alternatively, another element of the computer 120 in FIG. 1A, the display 130 in FIG. 1A, or another apparatus connected to the controller 122, may include one or more interfaces (not shown) such as a feedback interface to send data back to the clinician. An example of feedback that may be provided to a clinician via the controller 122 is haptic feedback such as a vibration or tone that warns the clinician that movement of the tissue has exceeded a predetermined threshold. Thresholds for movement may be translational and/or rotational. Exceeding a threshold of movement may trigger a warning for the clinician.

FIG. 1C illustrates an operational progression for sensors in dynamic tissue imagery updating, in accordance with a representative embodiment.

The operational progression for sensors in FIG. 1C may be representative of how inertial sensors are used in a clinical workflow for dynamic tissue imagery updating. In FIG. 1C, the organ is a lung, though dynamic tissue imagery updating as described herein is not limited to lungs as an application.

As shown in FIG. 1C, first sensor 195 to the fifth sensor 199 are placed on soft tissue of the lung as an organ at S110. In various embodiments, more or fewer than five sensors may be used, without departing from the scope of the present teachings. The five sensors are used to track deformation of the tissue of the organ. The five sensors may be small inertial sensors each with an integrated gyroscope and/or accelerometer. The five sensors are adhered or otherwise attached to the soft tissue of the organ at locations around an area of interest.

At S120, the five sensors are registered to pre-operative imagery. Registration involves aligning a three-dimensional coordinate system of the five sensors with a disparate three-dimensional coordinate system of the pre-operative imagery to provide a common three-dimensional coordinate system such as by sharing a common origin and set of axes. Registration will result in current locations of the five sensors being aligned at the corresponding locations in or on the organ in the pre-operative imagery. The pre-operative imagery may be, for example, optical imagery, magnetic resonance imagery, computed tomography (CT) imagery, or X-ray imagery. The pre-operative imagery may have been captured immediately before or after the placement of the five sensors at S110.

At S130, the five sensors begin streaming data. The five sensors individually each emit a signal that, collectively, are a set of electronic signals that include position vectors of positions of the five sensors. The five sensors iteratively emit sets of electronic signals that reflect movements of the five sensors between each set. The position vectors may each include three coordinates in the common three-dimensional coordinate system of the five sensors and the pre-operative imagery after the registration at S120.

The streaming at S130 may be by BLUETOOTH® and may be received at a receiver (not shown) located proximate to the five sensors, such as in the same operating room. The receiver that receives streamed data from the five sensors may provide the streamed data directly for processing to a controller 122 in FIG. 1A described above. Alternatively, the receiver may provide the streamed data directly for processing to a device or system that includes the controller 122, such as the computer 120 or another component of the system 100 in FIG. 1A. The receiver may be a component of the computer 120 in FIG. 1A. Alternatively, the receiver may be a peripheral device directly or indirectly connected to the computer 120.

The data streamed by each sensor at S130 may include a position vector of the position of the sensor, mentioned above, along with an identification of the sensor such as an identification number unique to the sensor. For example, each of the first sensor 195 to the fifth sensor 199 may stream a position vector and identification number. The coordinates of the position vectors in the common three-dimensional coordinate system may be based on readings from a gyroscope, an accelerometer, a compass, and/or one or more other components of each sensor. The position vectors and any other data from each sensor of the five sensors are sent in real-time via the streaming at S130.

At S140, the pre-operative imagery is updated to reflect a current state of the tissue of the organ. The updating at S140 is based on the data from the five sensors and reflects movement of the sensors that may be recognized from the position vectors in the data from the five sensors. The updating at S140 may be performed iteratively to morph the pre-operative imagery into a progressive series of updated imagery. Updated imagery, as the term is used herein, may refer to any iteration of updates starting from the original pre-operative imagery. Each update performed at S140 may result in a new iteration of updated imagery.

At S150, reference positions of the five sensors are obtained from the data streamed at S130. Since the five sensors are registered to the pre-operative imagery at S120 in the common three-dimensional coordinate system, the reference positions at S150 are obtained in the same coordinate space as the updated imagery that was updated at S140.

After obtaining the reference positions at S150, the process returns to S140 to iteratively update the pre-operative imagery again. That is, the reference positions obtained at S150 are used in the next iteration of S140 to further update the pre-operative imagery. The five sensors may stream data at S130 continually even as S140 and S150 are performed. The process of S140 and S150 may be performed in a loop that includes updating pre-operative imagery to become updated imagery, and then again newly obtaining reference positions of the five sensors for the next updating of the pre-operative imagery. As mentioned above, the streaming at S130 may be performed the entire time that the processes of S140 and S150 are initially and then subsequently performed in a loop. Each time the positions of the first sensor 195 to the fifth sensor 199 are newly obtained at S150 based on newly received data streamed by the first sensor 195 to the fifth sensor 199 at S130, the pre-operative imagery of the most recently updated imagery may be newly updated at S140. Accordingly, when the tissue of the organ moves, the corresponding position vector for each sensor may be obtained in real-time, and the pre-operative imagery may be updated in real-time.

FIG. 1D is a flow diagram illustrating a method for dynamic tissue imagery updating for the operational progression for sensors in FIG. 1C, in accordance with a representative embodiment.

The steps in the method of FIG. 1D correspond to the steps of the operational progression for sensors of FIG. 1C, as indicated by the reference numbers. At S110, sensors are placed on soft tissue of an organ. At S120, the sensors are registered to pre-operative imagery of the organ, such as pre-operative images from a computed tomography (CT) system or from an X-ray system. At S130, sensor data is streamed from the sensors. The sensor data may be continually streamed at S130 even as subsequent steps S140 and S150 are performed in a loop. At S140, pre-operative imagery is updated into updated imagery to reflect the current state of tissue. At S150, reference positions of the sensors are obtained. The reference positions obtained at S150 are in a common three-dimensional coordinate system for the sensors and the pre-operative imagery based on the registration at S120. After S150 the method of FIG. 1D may be performed in a loop between S140 and S150 as the sensor data is continually streamed at S130. In each iteration of the loop, the result of the previous iteration of the updating of pre-operative imagery at S140 is indicated as a reference position at S150 and is again updated based on the next set of electronic signals streams from the set of sensors.

As illustrated in the operational progression for sensors of FIG. 1C and explained with respect to the method of FIG. 1D, a mismatch between pre-operative imagery and a current state of tissue of an organ may be remedied by updating the pre-operative imagery of an organ based on movement of sensors that are registered to the pre-operative imagery of the organ. The position vectors from the sensors streamed at S130 may be used to create a real-time model of the sensors in three-dimensional space, which in turn may be used to morph the pre-operative imagery into updated imagery at S140. The real time modeling of the geometry of the sensor positions enables the updating of pre-operative imagery to reflect the current state of the tissue of the organ, thus eliminating the mismatch between the pre-operative images and the current state of the tissue of the organ. Examples of how the morphing may be performed are explained below.

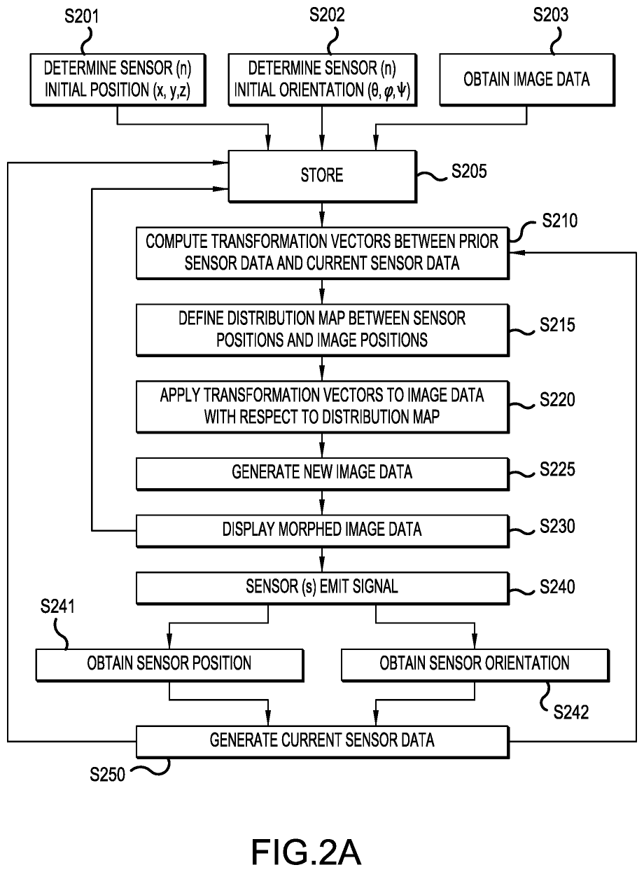

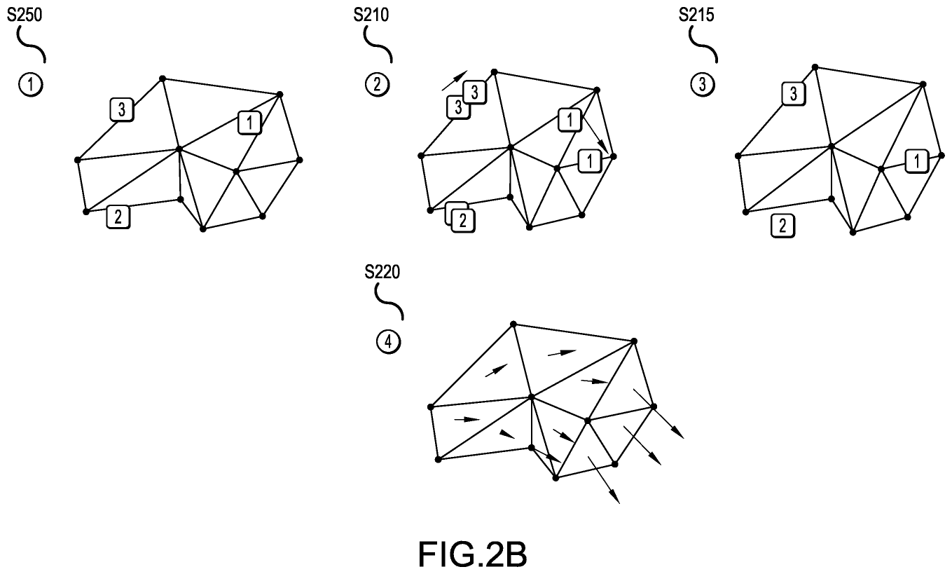

FIG. 2A illustrates another method for dynamic tissue imagery updating, in accordance with a representative embodiment.

The method of FIG. 2A starts at S201 by determining an initial position in three dimensions (x, y, z) for each sensor (n). The determinations at S201 may be performed by each sensor (n) (e.g., first sensor 195 to the fifth sensor 199), and/or may be performed by a processor that processes the sensor information from each sensor (n). At S202, an initial orientation (Θ,ϕ,ψ) is determined relative to each of three axes for each sensor (n). The determinations at S202 may also be performed by each sensor (n), and/or may also be performed by a processor that processes the sensor information from each sensor (n). The three dimensions may each be perpendicular to planes that include the other two dimensions. For instance, a first plane may be formed to include the y direction and the z direction, and the x direction is perpendicular to the first plane. A second plane may be formed to include the x direction and the z direction, and the y direction is perpendicular to the second plane. A third plane may be formed to include the x direction and the y direction, and the z direction is perpendicular to the third plane.

At S203, image data is obtained, such as by receiving the image data over a communications connection such as a wired or wireless connection. The image data obtained at S203 may be pre-operative image data that includes the soft tissue on or at which the sensors are placed. The image data obtained at S203 may be of anatomy that includes an organ and that may be obtained by CT imaging. S203 may be performed before the sensors are placed at or on the organ, so before S201 and S202. S203 may also be performed with the sensors already placed at or on the organ, so after S201 and S202.

At S205, the data of the initial position and initial orientation for each sensor (n) is stored along with the image data obtained at S203. The sensor data and the image data may be stored together in a memory such as the memory 12220 of FIG. 1B for processing by a processor such as the processor 12210 of FIG. 1B.

At S210, a transformation vector reflecting changes in positions and/or orientations between prior sensor data and current sensor data is computed for each sensor. The transformation vector may include a difference between readings for all three dimensions (x, y, z) and for all three orientations (Θ,ϕ,ψ) for each sensor. The first transformation computed based on the initial position and initial orientation will show no movement since there are no comparable prior readings. However, each subsequent reading of dimensions and orientations for each sensor will be comparable to the immediately previous reading or other previous readings. The transformation vectors computed at S210 may contain, for example, six values for the change in each dimension and in each orientation between readings for each sensor. The transformation vectors reflect the movement of each sensor between readings.

At S215, the method of FIG. 2A includes defining a distribution map between sensor positions and image positions from the pre-operative imagery or the immediately previous updated imagery. The distribution map will map sensor positions in the common three-dimensional coordinate system of the sensors and current iteration of the imagery. The first distribution map will show the initial sensor positions relative to the pre-operative imagery, and successive distribution maps will show the current sensor positions relative to the updated imagery. The distribution map may also show movement of each sensor (n) from the previous position of each sensor (n) to the current position of the sensor (n) relative to the pre-operative imagery or the immediately previous updated imagery.

At S220, the transformation vectors are applied to the image data from the pre-operative imagery or the immediately previous updated imagery. The application of transformation vectors involves adjusting the pre-operative imagery or the immediately previous updated imagery based on movement of the sensors from the previous sensor positions to the current sensor positions. The adjustment of pre-operative imagery or immediately previous updated imagery away from the previous sensor positions may be adjusted in relative correspondence to movements of the sensors. However, movement of the pre-operative imagery or immediately previous updated imagery may involve more than moving a single pixel of the pre-operative imagery or immediately previous updated imagery. For example, the transformation vectors may be applicable to entire fields of pixels in the pre-operative imagery or immediately previous updated imagery. The fields of pixels may be moved uniformly, such as when only one sensor (n) is used to track movement. The pixels within a field may also be moved non-uniformly, such as based on averages of movements of each of the closest two or three or four sensors (n) in each of the directions (x, y, z) and the orientations (Θ,ϕ,ψ) of the closest two or three or four sensors (n) relative to the three axes. The pixels within a field may also be moved non-uniformly such as based on weighted averages of movements of each of the closest two or three or four sensors (n) in each of the directions (x, y, z) and the orientations (Θ,ϕ,ψ) of the closest two or three or four sensors (n) relative to the three axes. For example, the movement of the closest sensor may be weighted disproportionately compared to movements of other sensors when determining the movement of a pixel.

As will be understood in the context of adjusting pixel positions in imagery based on proximity to sensors, a larger quantity of sensors provides greater spatial resolution of the updated imagery resulting from the model. Therefore, the number of sensors used may reflect a trade-off between (i) lower spatial resolution, accuracy and simpler processing for fewer sensors and (ii) cost of and complexity of implementing more sensors. For example, the number of sensors may be optimized to provide a high level of certainty with respect to overall deformation without requiring too much computational power and without covering the surface of an organ so as to be unnecessarily obscured. The processing requirements for dynamic tissue imagery updating include both identification of movement of the set of sensors, and the more complex image processing to morph the pre-operative imagery and the updated imagery iteratively for each set of electronic signals indicating movement by the set of sensors.

At S225, new image data for updated imagery is generated, which reflects movement of the sensors determined from the sensor data. The updated imagery may be based on the application of the transformation vectors at S220 and may include pixel values for each pixel as moved based on the transformation vectors at S220 in the pre-operative imagery or the immediately previous updated imagery. For most pixels in the pre-operative imagery or the immediately previous updated imagery, the new image data generated at S225 may be an estimation of the impact of tissue movement determined from the movement of the sensors, such as based on averages or weighted averages of readings from the nearest sensors.

At S230, the morphed image data resulting from S225 is displayed. The morphed image data from S230 is also stored at S205. The morphed image data may be displayed, for example, along with or superimposed on endoscopic video on the display 130 in FIG. 1A.

At S240, each sensor (n) emits a new signal. The new signals include new information of the positions and orientations of each sensor. At S241, a position in each of the directions (x, y, z) for each sensor (n) is obtained from the new signal emitted at S240. At S242, orientations (Θ,ϕ,ψ) of each sensor (n) relative to the three axes are obtained based on the new signal emitted at S240.

At S250, current sensor data is generated. The current sensor data generated at S250 is stored at S205 and fed back for the computation of the transformation vectors at S210. S250 may include the same determinations as in S201 and S202, but for subsequent readings of the sensor data. Accordingly, S250 may include determining a position in three dimensions (x, y, z) for each sensor (n), and determining an orientation (Θ,ϕ,ψ) relative to each of three axes for each sensor (n). The determinations at S250 may be performed by each sensor (n), and/or may be performed by a processor that processes the sensor information from each sensor (n). Since each generation of the current sensor data at S250 is after the initial position and initial orientation are generated at S201 and S202, when the method of FIG. 2A returns to S210 from S250, there will be a previous set of readings of the coordinates and orientations of each sensor to compare with the current readings to compute the transformation vectors at S250. Therefore, after S250 is initially performed, the transformation vector is computed between the initial position and orientation from S201 and S202 and the initial generation of current sensor data at S250. The transformation vector computed at S210 reflects changes in position (x, y, z) and in orientation (Θ,ϕ,ψ) relative to the three axes.

FIG. 2B illustrates sensor movement for the method for dynamic tissue imagery updating in FIG. 2A, in accordance with a representative embodiment.

The model labelled as “1” in FIG. 2B corresponds to S250 (or S201) in FIG. 2A. This model corresponds to current sensor data generated based on the current sensor positions and orientations.

The model labelled as “2” in FIG. 2B corresponds to S210 in FIG. 2A. This model corresponds to transformation vectors computed between prior sensor data and the current sensor data generated at S250. As shown in this model, each sensor has shifted from the prior position to the current position. The movement of the sensors may be used to morph the last iteration of the pre-operative imagery or the updated imagery at S220.

The model labelled as “3” in FIG. 2B corresponds to S215 in FIG. 2A. This model corresponds to a distribution map defined between the current sensor positions and the positions of imagery of the last iteration of the pre-operative imagery or the updated imagery. In other words, this model shows the updated positions of the sensors compared to the imagery of the last iteration of the pre-operative imagery or the updated imagery, since the last iteration of the pre-operative imagery or the updated imagery has not yet been morphed based on the most recent movement of the sensors.

The model labelled as “4” in FIG. 2B corresponds to S220 in FIG. 2A. This model corresponds to transformation vectors applied to the image data of the imagery of the last iteration of the pre-operative imagery or the updated imagery. The transformation vectors of the sensors are used to update individual pixels from the last iteration of the pre-operative imagery or the updated imagery, such as using averages or weighted averages of movement of the nearest sensors in each of three directions and in each of three orientations. The arrows roughly show directions of movement of the tissue. The movement of each pixel from the most recent version of the pre-operative imagery or the updated imagery may be weighted by proximity of the pixel to the sensor in each of the three directions (x, y, z). As a result, the closer the pixel is to any sensor, the more strongly movement of that sensor will reflect movement of the pixel. Since the tissue of an organ will move as a whole in addition to moving at individual points, each iteration of updates from the pre-operative imagery will appear as a smooth change in positions of the tissue of the organ.

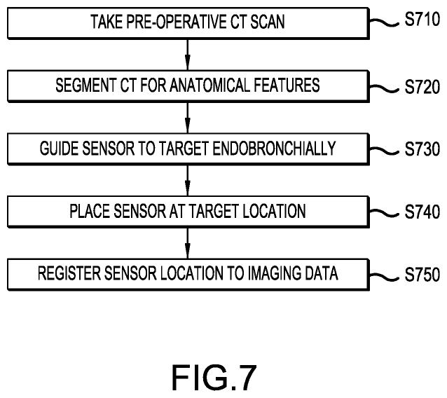

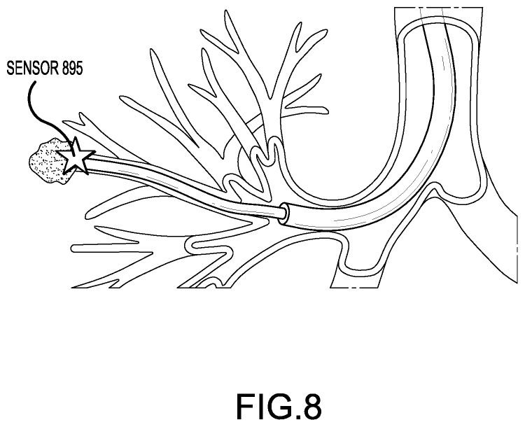

Embodiments described herein largely use lung surgery as an example use case, but the dynamic tissue imagery updating applies equally to other procedures involving highly deformable tissue, such as, but not limited to liver and kidney surgery. Additionally, embodiments herein largely describe placement of sensors on the surface of an organ, but sensors may also be placed inside an organ via a lumen access or a percutaneous needle access in some embodiments. For example, the use of internal sensors may be introduced endobronchially in the lung as shown in the embodiment of FIG. 7 explained below. As another example, internal sensors may also be introduced through the blood vessels for the kidney.

Sensors on the surface of an organ may be more readily detected in imagery, while sensors in the interior of an organ may better localize tumors, vessels, and airways due to the proximity of the sensors to these structures. Sensors on the surface may instead be correlated to surface features, which may be valuable when the surface features are detectable in other modalities, such as MRI, CT, CBCT or X-ray, to facilitate registration between the modalities. Lung fissures represent one possible use for surface sensors.

FIG. 3 illustrates a sensor for dynamic tissue imagery updating, in accordance with a representative embodiment.

As shown in FIG. 3, a sensor 300 includes an adhesive pad 310, a battery 320, a transmitter 330, and an ASIC 340 (application-specific integrated sensor). The sensor 300 is an example of an inertial sensor for surgical use. The sensor 300 may be disposable or re-usable. Additionally, the sensor 300 may be sealed by a sterile protective casing (not shown) that may be biocompatible. Such a sterile protective casing may enclose and seal the battery 320, the transmitter 330, the ASIC 340 and other components provided in the sensor 300.

The adhesive pad 310 may be a biocompatible adhesive and is configured to adhere the sensor 300 to the organ or other area of interest. The adhesive pad 310 may be adhered to a surface of a sterile protective casing (not shown) that encloses and seals other components of the sensor 300. Alternatively, the adhesive pad 310 may form a lower surface of a sterile protective casing. The adhesive pad 310 is representative of mechanisms for attaching the sensor 300 to the organ or other area of interest. Alternatives to the adhesive pad 310 that may be used to attach the sensor 300 to tissue include an eyehole for receiving a suture or a mechanism for receiving a staple, which attach the sensor 300 in FIG. 3 directly to tissue.

The battery 320 serves as a power source for the sensor 300, such as a disposable coin cell battery, for example. The battery 320 supplies power to one or more components of the sensor 300, including the transmitter 330, the ASIC 340 and other components. Alternatives to the battery 320 include mechanisms for receiving power from an external source. For example, a photodiode provided to the sensor 300 may be powered from an external source such as light from the interventional imagery source 110. A power source that includes the photodiode and a storage device such as a capacitor may be energized by light from the interventional imagery source 110. For example, light from the interventional imagery source 110 hitting a photodiode in the sensor 300 may be used to charge a capacitor in the sensor 300, and the power from the capacitor may be used for other functionality of the sensor 300. Additional methods of powering a sensor 300 may include converting external energy sources into power for the sensor 300, such as capturing heat from electrocautery tools or a sound wave from an ultrasound transducer.

The transmitter 330 is a data transmitter for transmitting position and orientation data of the sensor 300. The transmitter 330 may be a BLUETOOTH® transmitter, for example.

The ASIC 340 may include circuitry such as gyroscope circuitry implemented on a circuit board along with any other circuit elements needed for any other positional and rotational functions. The ASIC 340 collects data for determining absolute positions and/or relative positions of the sensor 300. The ASIC 340 may be a combined gyroscope and electronics board. Additional components that may be used in the sensor 300 to determine absolute positions and/or relative positions of the sensor 300 include an accelerometer and a compass, which may be integrated on the electronics board of the ASIC 340.

One instantiation of the sensor 300 may be used in some embodiments of dynamic tissue imagery updating. In other embodiments multiple instantiations of the sensor 300 may be used. Multiple instantiations of the sensor 300 provided together in a configuration may be self-coordinated, such as by logic provided to each of the multiple instantiations of the sensor 300 to coordinate an origin and axes for a common coordinate system of the configuration. The common coordinate system of the configuration of multiple instantiations of the sensor 300 may be used for the registration with the pre-operative imagery. Logic provided to each of multiple instantiations of the sensor 300 may include a microprocessor (not shown) and a memory (not shown). In other embodiments, multiple instantiations of the sensor 300 provided together in a configuration may be coordinated externally, such as by the controller 122 of FIG. 1B alone, or by the controller 122 of FIG. 1A in the system 100.

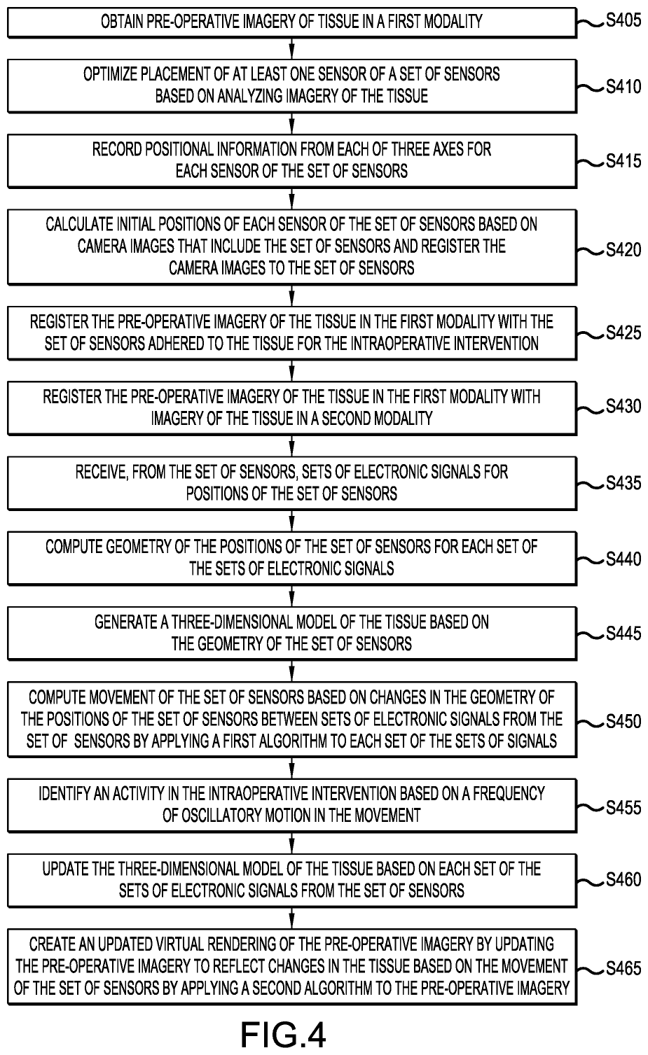

FIG. 4 illustrates another method for dynamic tissue imagery updating, in accordance with a representative embodiment.

The method of FIG. 4 starts at S405 by obtaining pre-operative imagery of tissue in a first modality. The pre-operative imagery of tissue may be obtained immediately before the medical intervention in which the dynamic tissue imagery updating is performed or may be performed well before the medical intervention. The pre-operative imagery may be, for example, CT imagery such that the first modality is CT imaging. A memory such as the memory 12220 may store the pre-operative imagery of tissue obtained in the first modality along with instructions such as software instructions to be executed by the processor 12210. Alternatively, the pre-operative imagery of tissue obtained in the first modality may be stored in a first memory and the software instructions may be stored in a second memory.

At S410, placement of at least one sensor of a set of sensors is optimized based on analyzing imagery of the tissue. The set of sensors includes one or more sensors for the entirety of an instantiation of the dynamic tissue imagery updating described herein. When there is only one sensor, the location to place the sensor may be optimized based on the circumstances of the medical intervention in which the single sensor is to be placed. For example, the single sensor may be placed next to tissue that is to be removed when only a small amount of tissue is to be removed. Alternatively, when there are multiple sensors, the placement of multiple sensors may be optimized in a configuration at S410. The use of multiple sensors improves refinement of the updated imagery while imposing greater processing requirements. For example, multiple sensors may be placed around a mass of tissue that is to be removed from an organ.

The optimization at S410 may be based on machine learning applied to previous instantiations of sensor placement in medical interventions. For example, the machine learning may have been applied at a central service that receives imagery and details from geographically diverse locations in which the previous instantiations of sensor placement are performed. The machine learning may also have been applied in a cloud, such as at a data center. The optimization may be applied at S410 based on the results of the machine learning, such as by using an algorithm generated or retrieved specifically for the circumstances of the medical intervention in which the optimized placement of sensors at S410 will be used. An algorithm for the optimization at S410 may include customized rules based on the type of medical intervention, the medical personnel involved in the medical intervention, characteristics of the patient subjected to the medical intervention, previous medical imagery of the tissue involved in the medical intervention, and/or other types of details that may result in varying what is considered an optimal sensor placement.

At S415, the method of FIG. 4 includes recording positional information from each of three axes for each sensor of the set of sensors. The positional information may reflect the same common three-dimensional coordinate system. The set of sensors may be, for example, self-coordinated to set a common origin and three axes. One or more sensors of the set of sensors may be equipped to measure signal strength from signals from the other sensors in order to determine relative distance in each direction of the other sensors. Alternatively, the set of sensors may be, for example, externally coordinated to set the origin and the three axes in common, such as by the controller 122 of FIG. 1B alone or in the system 100 of FIG. 1A. For example, signal strength may be received externally, such as by an antenna connected to the controller 122, and the signal components in each direction may be used to determine relative distance in each direction of each of the set of sensors.

In an embodiment in which the set of sensors are self-coordinated, one of the set of sensors may be set as the common origin for a common three-dimensional coordinate system. When the set of sensors are self-coordinated, the sensors may be provided with logic such as a memory that stores software instructions and a processor (for example, a microprocessor) that executes the instructions. In some embodiments, the sensors themselves may contain circuitry for positional tracking such as via electromagnetic tracking which provides a coordinate system for the sensor(s). In this case the set of sensors may be aligned with one another prior to the interventional procedure or as a registration step during the interventional procedure. In an embodiment, the set of sensors may be placed in a predetermined pattern that maintains a specific predetermined orientation with respect to one another. For example, a first sensor may always be placed on the upper left lobe, a second sensor may always be placed on the lower left lobe of the lung, and a third sensor may be placed in an area that will not be subject to movement. In this embodiment, a standard pattern of placement for sensors may ensure a uniform starting position with a reference to a fixed sensor with a known position in the imagery. When self-coordinated, each sensor may be aware of its position in the common three-dimensional coordinate system.

When coordinated from outside, such as by the controller 122, the sensors do not have to be aware of their positions in the common three-dimensional coordinate system and may instead simply report translational and rotational changes in position to the controller 122. When coordinated from outside, such as by the controller 122, each sensor in a set of sensors may use its initial location as an origin in its own three-dimensional coordinate system, and the controller 122 may adjust each set of sensor data received from the sensors to offset the original location of each sensor from the origin of the common three-dimensional coordinate system set for the sensors. Using the operational progression of FIG. 1C as an example, each of the five sensors in FIG. 1C may have its own coordinate system derived from the same types of readings, such as based on readings of gravity as the Y direction by an accelerometer, true north by a compass as the Z direction, and derivation of an X direction perpendicular to a plane that includes the Y direction and the Z direction. The recorded positional information may therefore show comparable initial coordinates for each sensor of the set of sensors. The sensor data from the set of sensors may therefore be adjusted to a common three-dimensional coordinate system, such as by a controller 122 from FIG. 1B alone or in the system 100 of FIG. 1A.

At S420, the method of FIG. 4 includes calculating initial positions of each sensor of the set of sensors based on camera images that include the set of sensors and registering the camera images to the set of sensors. The initial positions of each sensor may be supplemental or alternative to the recording of positional information at S415. The camera that provides the camera images can be a traditional camera with a two-dimensional (2D) view or a stereo camera with a three-dimensional view. The initial positions of each sensor calculated at S420 may be set in a space defined from the view of the camera as the origin of a common three-dimensional coordinate system for the sensors. When S415 and S420 supplement each other, the common three-dimensional coordinate system for the positional information recorded at S415 may be adjusted to match the common three-dimensional coordinate system for the initial positions of each sensor calculated at S420. As a result, the initial positions of each sensor calculated at S420 may be a second set of positions of the sensors and may be calculated in order to register the recorded positional information from S415 with the positional information calculated from the imagery at S420. More than one image may be taken for the calculations at S420 to improve the registration in the common three-dimensional coordinate systems of S415 and S420 or in the case when a sensor is not seen by the camera. Rotating the camera to another position may enable detection of the position of the sensor. Two two-dimensional camera views may be used with a back projection method to identify the three-dimensional position of each of the set of sensors in the two-dimensional images. When performed as an alternative to S415, the common three-dimensional coordinate system of the initial positions calculated based on camera images at S420 may be imposed as the common three-dimensional coordinate system on the set of sensors. When the sensors are informed of their coordinates and the three axes in a common three-dimensional coordinate system, sensor data from the sensors may be accurate positional and rotational information in the common three-dimensional coordinate system. Alternatively, the sensors may not be aware of their coordinates and/or the three axes in the common three-dimensional coordinate system for the calculated initial positions at S420, in which case a controller 122 from FIG. 1B alone or in the system 100 of FIG. 1A may adjust the positional and rotational information in the sensor data into the common three-dimensional coordinate system for the sensors.

As the tissue moves and hence the sensors move the coordinates of the sensors can be adjusted using the inertial data streamed from each sensor. The registration between the common three-dimensional coordinate systems for S415 and S420 may be updated during the procedure by acquiring new camera views. A stereo camera may also be used for improved three-dimensional registration. In other embodiments, electromagnetic sensing or compass data may be used for the calculation of initial sensor positions at S420.

At S425, the method of FIG. 4 next includes registering the pre-operative imagery of the tissue in the first modality with the set of sensors adhered to the tissue for the interventional medical procedure. The registration may involve one or both of the common three-dimensional coordinate systems generated at/for S415 and S420, along with the coordinate system of the pre-operative imagery. Registration may be performed by aligning landmarks in the pre-operative imagery with the placement of the sensors in or on the tissue that was previously subject to the pre-operative imagery, whether derived from logical control at S415 or from camera imagery at S420.

In calculating initial positions of each sensor and registering the camera images to the set of sensors at S420, the camera images may also be registered to the pre-operative imagery. As another alternative to the registration based on S415, S420 and S425, registration may be performed by placing the sensors on the tissue and then acquiring the pre-operative imagery. The positions and orientations of the sensors can be extracted from the pre-operative imagery with respect to the anatomy in the imagery. This may avoid a requirement for a direct camera view of the sensors on the organ as in S420.

Once the registration at S425 takes place, movement of the tissue that results in movement of the sensors can be tracked in the common three-dimensional coordinate system(s) for the sensors initially set at S415 and/or S420. The controller 122 may continually adjust sensor information from each sensor to the common three-dimensional coordinate system(s) as sensor data is received from each sensor. The registration at S425 may result in the pre-operative imagery being assigned initial coordinates in the common three-dimensional coordinate system(s) set for the sensors at S415 and/or S420.

At S430, the method of FIG. 4 includes registering the pre-operative imagery of the tissue in the first modality with imagery of the tissue in a second modality. Returning to the example of the operational progression of FIG. 1C, the coordinate system of the pre-operative imagery may be partially or fully defined in the pre-operative imagery, and once registered at S425 may be set in the common three-dimensional coordinate system of the set of sensors. For example, landmarks in the pre-operative imagery may each be assigned coordinates in three directions and rotations about three axes in the common three-dimensional coordinate system of the set of sensors. Thus, the coordinate system of the pre-operative imagery may also be that of the common three-dimensional coordinate system for the sensor set at S420, based on the registration at S425.

For S430, insofar as anatomical features may be detectable in one or more second modalities such as by X-ray or endoscope/thoracoscope, when one or more sensors are placed adjacent to anatomical features the sensors can be registered to the one or more second modalities. Imagery from the second modalities may be registered with positions in the common three-dimensional coordinate system for the sensors as set at S415 and/or S420. For example, when endobronchial sensors are placed adjacent to a tumor and in at least two other airways, the endobronchial location and the other two airways may be found in a segmented CT image and the segmented CT image can be registered to the common three-dimensional coordinate system for the sensors. Additionally, registration as at S425 and at S430 may be possible with fewer than three sensors by predefining placement locations of the sensors, or by incorporating data of past procedures.

At S435, the method of FIG. 4 includes receiving, from the set of sensors, sets of electronic signals for positions of the set of sensors. As noted, the electronic signals may include sensor data already set in the common three-dimensional coordinate system, or may be adjusted, for example by the controller 122 in FIG. 1B, to fit the common three-dimensional coordinate system.

At S440, the method of FIG. 4 includes computing geometry of the positions of the set of sensors for each set of the sets of electronic signals. The geometry may include the individual sensor positions in the common three-dimensional coordinate system of the sensors, as well as relative differences in coordinates between positions of different sensors. The sets of electronic signals from the sensors are input to an algorithm at a controller 122. The controller 122 may continuously compute the geometry of the sensors relative to the tissue of the organ.

The geometry computed at S440 may include positioning of one sensor in the common three-dimensional coordinate system, as well as movement of each sensor in the common three-dimensional coordinate system over time. The geometry may also include positioning of each of multiple sensors in the common three-dimensional coordinate system, relative positioning of the multiple sensors in the common three-dimensional coordinate system, and movements of the positioning and relative positioning of the multiple sensors over time.

At S445, the method of FIG. 4 includes generating a three-dimensional model of the tissue based on the geometry of the set of sensors. The three-dimensional model generated at FIG. 4 may be an initial three-dimensional model of the positions of the set of sensors in the common three-dimensional coordinate system with features corresponding. For example, the three-dimensional model may be restricted to information of the geometry of the sensors and may exclude the pre-operative imagery.

At S450, the method of FIG. 4 includes computing movement of the set of sensors based on changes in the geometry of the positions of the set of sensors between sets of electronic signals from the set of sensors by applying a first algorithm to each set of the sets of signals. The movement may be reported in transformation vectors including three sets of translational data for movement in each of the three directions, and three sets of rotational data for movement about each axis. The movement may be continually computed during the medical intervention after the sensors are placed.

At S455, the method of FIG. 4 includes identifying an activity during an interventional medical procedure based on a frequency of oscillatory motion in the movement. The activity identified at S455 may be identified based on pattern recognition, such as a particular frequency of oscillation of a sensor that is known to correspond to a particular type of activity that occurs during medical interventions.

At S460, the method of FIG. 4 includes updating the three-dimensional model of the tissue based on each set of the sets of electronic signals from the set of sensors. The three-dimensional model of the tissue is updated at S460 to first show movement of the sensors computed at S450. The updated model may identify the current position of each sensor in the common three-dimensional coordinate system and may identify one or more previous positions of each sensor to reflect the relative movement of each sensor over time.

At S465, an updated virtual rendering of the pre-operative imagery is created by updating the pre-operative imagery to reflect changes in the tissue based on the movement of the set of sensors by applying a second algorithm to the pre-operative imagery. The pre-operative imagery is updated at S465 to morph the pre-operative imagery by moving each pixel from the previous iteration of the pre-operative imagery by amounts corresponding to the movement of the sensors. Individual pixels may be moved by different amounts in different directions based on proximity to different sensors that move in different amounts in different directions. The movement for each pixel in updated virtual rendering may be calculated based on averaging or weighted averaging of movement in each direction of the nearest sensor(s).

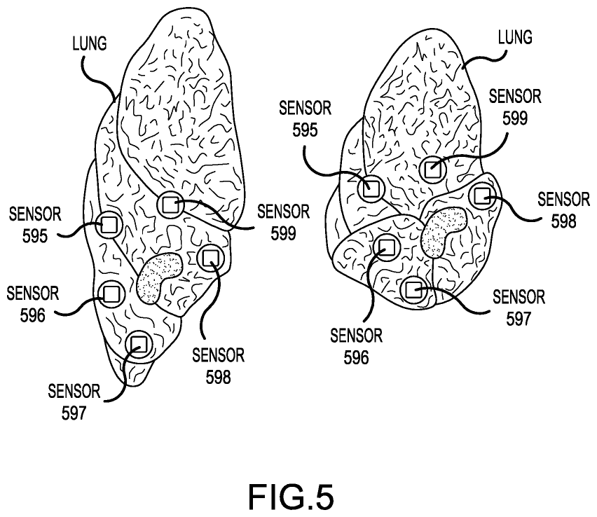

FIG. 5 illustrates another operational progression for sensors in dynamic tissue imagery updating, in accordance with a representative embodiment.

In FIG. 5, five sensors are placed on a lung. The five sensors include a first sensor 595, a second sensor 596, a third sensor 597, a fourth sensor 598 and a fifth sensor 599. The lung is only an example of an organ or other mass of tissue that can be subject to dynamic tissue imagery updating as described herein. The five sensors may be placed by adhesion, stapling, or sutures, for example. When placed by adhesion, for example, the five sensors may each be attached to the soft tissue with surgically compliant adhesive on the bottom of each sensor.

In FIG. 5, the five sensors are placed around a tumor or region of interest that was identified in pre-operative imagery from pre-operative imaging such as CT imaging. The exact number and locations of the sensors is not necessarily critical, as the deformable tissue model may adapt to levels of input data that vary based on the number and locations of the sensors. In FIG. 5, the left image shows the five sensors around a tumor in a collapsed lung, and the right image shows the backs of the five sensors as the lung is flipped by a clinician. In dynamic tissue imagery updating as described herein, the flipping of the lung can be detected from the model of the sensors as the set of sensors flip with the lung. In the example of FIG. 5, the second sensor 596 and the third sensor 597 are out of view in the right image due to being placed on the tissue of the lung that is in view in the left image but which is flipped in the right image.

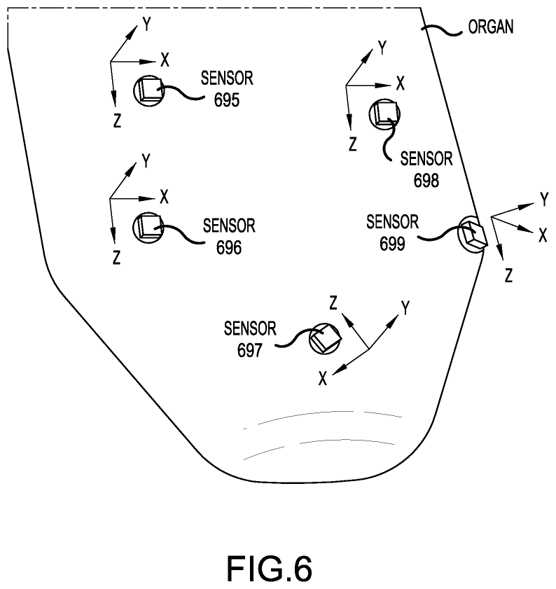

FIG. 6 illustrates an arrangement of sensors on tissue in dynamic tissue imagery updating, in accordance with a representative embodiment.

In FIG. 6, five sensors are placed on an organ. The five sensors include a first sensor 695, a second sensor 696, a third sensor 697, a fourth sensor 698, and a fifth sensor 699. The five sensors in FIG. 6 are adhered to the outside of the organ. The local coordinate systems for each of the five sensors in FIG. 6 are visualized for reference. Each sensor may use an internal position of the sensor as the origin of its local coordinate system. Position information from all three axes for each of the five sensors may be recorded by gyroscopes, for example, for each individual sensor and transmitted in real-time back to a central receiver, such as in an operating room.

Although the location for each of the five sensors in FIG. 6 may be placed randomly, the exact location for each sensor may also be optimized by identifying manually or automatically tissue locations that are most susceptible to large amounts of motion/deformation. For example, the lung is most rigid near the large airways which contain significant amounts of collagen, while it is most deformable at the edges. In the case of a lung, it is therefore advisable to attach one or more of the five sensors near the edge of the lung. The exact location can be determined from an algorithm applied to the pre-operative images or from the surgical view (eye or camera) or may be some combination of both.

FIG. 7 illustrates another method for dynamic tissue imagery updating, in accordance with a representative embodiment.

The method in FIG. 7 is a workflow appropriate, for example, to placing an inertial marker endobronchially as in FIG. 8 which is explained below. The workflow prepares a location for the sensor for image guidance.

The method in FIG. 7 starts at S710 with taking a pre-operative CT, MR, or CBCT scan. At S720, the method of FIG. 7 includes segmenting the pre-operative imagery for anatomical features. Segmentation is a representation of the surface of structures such as anatomical features such as the organ in FIG. 6 and consists for example of a set of points in three-dimensional (3-D) coordinates on the surfaces of the structure, and triangular plane segments defined by connecting neighboring groups of three points, such that the entire structure is covered by a mesh of non-intersecting triangular planes.

At S730, a sensor is guided to a target endobronchially using the segmented representation of anatomy from S720 as a reference for the path to the target. At S740, the sensor is placed at the target location. At S750, the sensor location is registered to imaging data. As noted above, the method of FIG. 7 is a workflow appropriate, for example, to placing an inertial sensor endobronchially as in FIG. 8.

FIG. 8 illustrates a sensor placement in dynamic tissue imagery updating, in accordance with a representative embodiment.

In the example of FIG. 8, a sensor 895 is a single inertial sensor and may be introduced into the lung endobronchially through airways before or during surgery. The sensor 895 may be advantageously placed as close to the tumor as possible, or near a major airway, blood vessel, or other distinct anatomical feature. The placement of the sensor 895 allows the sensor 895 to be directly localized relative to the target anatomy.

The placement procedure for the sensor 895 in FIG. 8 may leverage existing methods for endobronchial navigation, and may include endobronchial catheters guided by bronchoscopy, X-ray, CT, or electromagnetic (EM) tracking. The initial position of the sensor 895 may be registered to a thoracoscope or another type of endoscope for use in continuous tracking of the position of the sensor 895. The sensor 895 may be attached to the anatomy by leaving the sensor 895 in place (thereby relying on tissue support), anchoring the sensor 895 with barbs, clipping the sensor 895 to tissue, and/or adhering the sensor 895 to tissue using glue, for example. In the example of FIG. 8, once the sensor 895 is placed then intraoperative states of the lung tissue are interpreted based on readings such as orientation and motion from components of the sensor 895 such as an accelerometer.

The data from tracking the sensor 895 may include orientation of the sensor 895, and the data may be used to morph a lung model such as by recording the orientation of the sensor 895 relative to gravity (as a reference coordinate system) at the time the sensor 895 is placed. The corresponding initial orientation of the lung surface may be saved. The initial orientation may also be measured visually from thoracoscopic images or approximated from past procedures. As a result, changes in the orientation of the pertinent tissue can thus be tracked using the data from tracking the sensor 895. The orientation measurements from the sensor 895 may also be combined with other information sources, such as a biophysical model of the lung or tissue tracking in live video, to determine the location of the sensor 895 intraoperatively.

The data from tracking the sensor 895 may also be used to determine when the lung or another organ has been flipped. In this example, the orientation of the accelerometer in the sensor 895 may be used to determine whether the lung has been flipped, that is, which surface of the lung tissue is visible in the thoracoscopic view. For example, the orientation of the sensor 895 may be used to determine that the anterior or posterior of the lung is visible, that the inferior or superior of the lung is visible, and/or that the lateral or medial of the lung is visible in the thoracoscopic view. The capability to determine positioning of the lung may be useful in informing the clinician as to which surface of the lung is visible and may be further used to supplement image processing algorithms on/for the thoracoscope.

The data from tracking the sensor 895 in FIG. 8 may also be used to determine velocity and acceleration. Motion profiles measured by the accelerometer of the sensor 895 may be used to find motion patterns that correspond with various surgical events, such as dissection, incision, flipping, stretching, and manipulation. For example, oscillatory motion on the order of 0.5 Hz may indicate that dissection is occurring. Higher frequency motion on the order of 10 Hz may indicate that the stapling is occurring. These motion patterns may be further combined with other information sources, such as live video or instrument tracking, to enhance interpretation of surgical events.

Inertial sensor data may be used in real-time as described herein. For example, accelerometer data may be further analyzed for inertial tracking to determine location in real-time. The accelerometer data may be similar to types of information described already and can be incorporated into various forms of surgical guidance. For example, accelerometer data may be used to show a clinician a virtual model of the lung, deformed according to the real lung, based on accelerometer measurements. The location of the tumor or other anatomical features may be simultaneously superimposed depending on the placement of the sensor 895. In another example, accelerometer data can be used to show a clinician a visual (e.g., video) of the real lung, while simultaneously superimposing a virtual representation of the tracked sensor 895 and/or associated anatomical features. In yet another example, accelerometer data can be used to present to the clinician other forms of information or statistics, such as the distance the tumor has moved from its initial location, or the types of surgical events that have been detected. Recording this information can be used for marking the location and number of lymph nodes dissected.

In the examples of use for accelerometer data described above, the sensor 895 may be a single accelerometer-based sensor and may be used to create guidance that is advantageous for lung surgery. Single sensor solutions may be simpler to deploy and may be more cost effective than multi-sensor renditions. On the other hand, multi-sensor solutions bear several advantages including providing higher fidelity tracking of the deformable tissue or when using multiple independent sensors. Alternatively, multiple sensors in a known, fixed configuration allows the sensors to be registered to the tissue or thoracoscope without an explicit user-initiated registration step, such as using image based sensor detection, which may simplify the workflow.

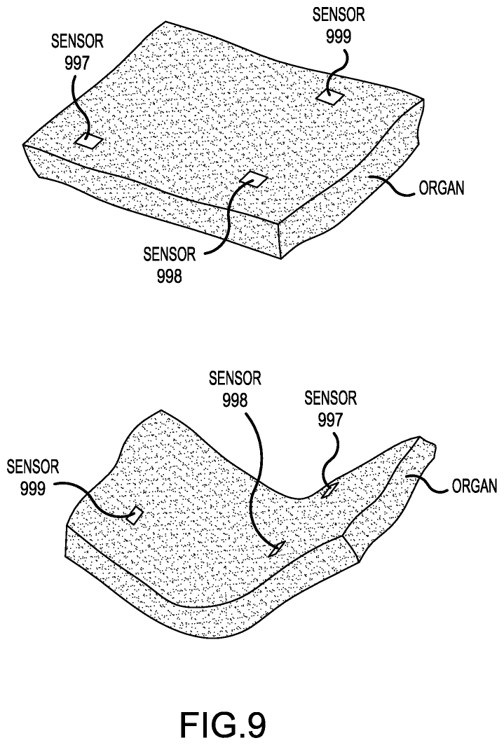

FIG. 9 illustrates another operational progression for sensors in dynamic tissue imagery updating, in accordance with a representative embodiment.

In FIG. 9, pre-operative imagery is morphed based on deformations detected from movement of sensors. Once the sensors are registered and initialized and the positions of the sensors is transmitted in real-time, the positions and orientations of the sensors can serve as input to an algorithm. The algorithm may work by starting with a pre-operative CT volume or three-dimensional volume of the target organ. This provides a static reference or starting place for the model. Input data from the sensors then provides individual position vectors for each sensor in real time. The position vectors may then be used to deform the pre-operative model, and thus predict the current state of the tissue in three-dimensional space. This new model may be refreshed at the same rate at which data is being transmitted from the sensors. In FIG. 9, deformation of an image may be based on algorithms as explained herein. FIG. 9 illustrates results of an experiment in which three commercial sensors were attached to the surface of a phantom. Accordingly, the three-dimensional model may be morphed based on the tracking of the sensors.

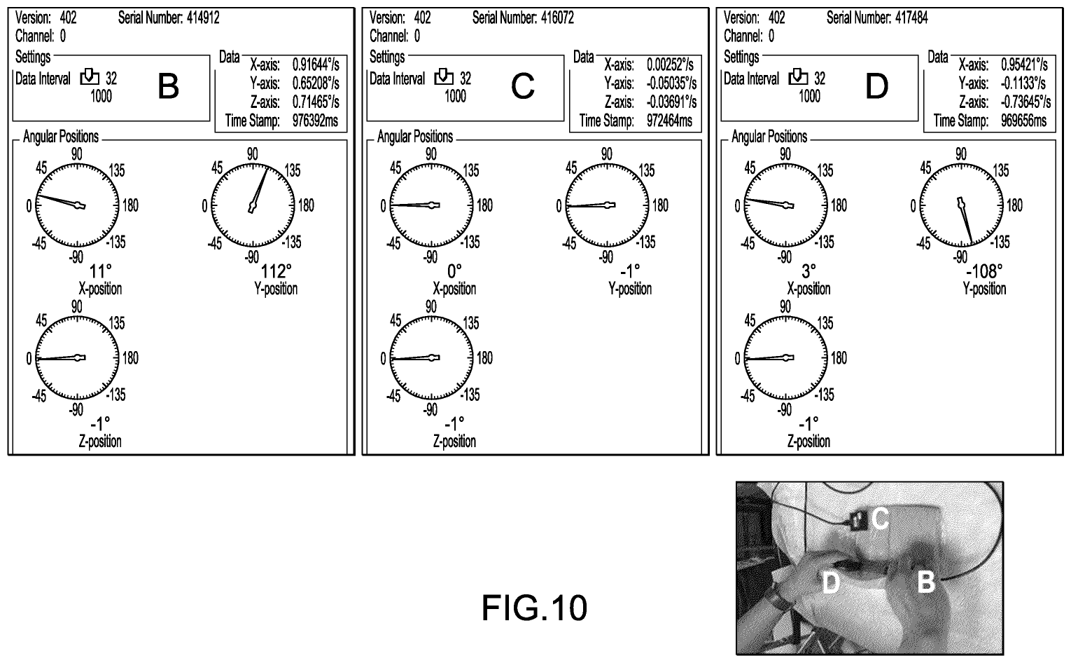

FIG. 10 illustrates a user interface for an apparatus monitoring sensors in dynamic tissue imagery updating, in accordance with a representative embodiment.

FIG. 10 illustrates an interface, such as a graphical user interface (GUI), that presents sensor data collected in real-time as a phantom that is flipped and rotated in various orientations. The three instantiations of the interface are labelled B, C and D, and each shows position readings in the three directions (x, y, z), time stamps for the readings, and angular positions relative to the three axes. The orientation of the sensors may be visualized as flat planes that may vary in colors such as green, blue, and yellow. These orientations are used to then morph the imagery of the tissue starting with the pre-operative imagery and through iterations of the updated imagery. Data from each of three sensors are illustrated in FIG. 10, including data after the tissue phantom is flipped.

In an alternative embodiment, haptic feedback may be applied via an interface, such as when movement exceeds a predetermined threshold. For example, information about the flipping or rotation of tissue may be provided to a clinician via haptic feedback that is provided from the controller 122 or another element of the computer 120 via a feedback interface. An example of haptic feedback may be a vibration sent to the clinician through a wearable device or a surgical tool when a sensor shows more than 90 degrees of rotation around any one-axis. An example of a feedback interface may be a port for a data connection, where the haptic aspect of the feedback is physically output based on data sent via the data connection. The threshold could be adjusted manually or automatically. Other forms of feedback may include light or sound visible as an external feature or within the thoracoscope camera view.

FIG. 11 illustrates a general computer system, on which a method for dynamic tissue imagery updating may be implemented, in accordance with another representative embodiment.

The general computer system of FIG. 11 shows a complete set of components for a communications device or a computer device. However, a “controller” as described herein may be implemented with less than the set of components of FIG. 11, such as by a memory and processor combination. The computer system 1100 may include some or all elements of one or more component apparatuses in an interactive endoscopic annotation system described herein, though any such apparatus may not necessarily include one or more of the elements described for the computer system 1100 and may include other elements not described.

The computer system 1100 can include a set of software instructions that can be executed to cause the computer system 1100 to perform any one or more of the methods or computer-based functions disclosed herein. The computer system 1100 may operate as a standalone device or may be connected, for example, using a network 1101, to other computer systems or peripheral devices. In embodiments, a computer system 1100 may be used to perform logical processing based on digital signals received via an analog-to-digital converter as described herein for embodiments.

In a networked deployment, the computer system 1100 may operate in the capacity of a server or as a client user computer in a server-client user network environment, or as a peer computer system in a peer-to-peer (or distributed) network environment. The computer system 1100 can also be implemented as or incorporated into various devices, such as a stationary computer, a mobile computer, a personal computer (PC), a laptop computer, a tablet computer, or any other machine capable of executing a set of software instructions (sequential or otherwise) that specify actions to be taken by that machine. The computer system 1100 can be incorporated as or in a device that in turn is in an integrated system that includes additional devices. In an embodiment, the computer system 1100 can be implemented using electronic devices that provide voice, video or data communication. Further, while the computer system 1100 is illustrated in the singular, the term “system” shall also be taken to include any collection of systems or sub-systems that individually or jointly execute a set, or multiple sets, of software instructions to perform one or more computer functions.

As illustrated in FIG. 11, the computer system 1100 includes a processor 1110. A processor for a computer system 1100 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A processor is an article of manufacture and/or a machine component. A processor for a computer system 1100 is configured to execute software instructions to perform functions as described in the various embodiments herein. A processor for a computer system 1100 may be a general-purpose processor or may be part of an application specific integrated circuit (ASIC). A processor for a computer system 1100 may also be a microprocessor, a microcomputer, a processor chip, a controller, a microcontroller, a digital signal processor (DSP), a state machine, or a programmable logic device. A processor for a computer system 1100 may also be a logical circuit, including a programmable gate array (PGA) such as a field programmable gate array (FPGA), or another type of circuit that includes discrete gate and/or transistor logic. A processor for a computer system 1100 may be a central processing unit (CPU), a graphics processing unit (GPU), or both. Additionally, any processor described herein may include multiple processors, parallel processors, or both. Multiple processors may be included in, or coupled to, a single device or multiple devices.

A “processor” as used herein encompasses an electronic component which is able to execute a program or machine executable instruction. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each including a processor or processors. Many programs have software instructions performed by multiple processors that may be within the same computing device or which may even be distributed across multiple computing devices.

Moreover, the computer system 1100 may include a main memory 1120 and a static memory 1130, where memories in the computer system 1100 may communicate with each other via a bus 1108. Memories described herein are tangible storage mediums that can store data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A memory described herein is an article of manufacture and/or machine component. Memories described herein are computer-readable mediums from which data and executable software instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

Memory is an example of a computer-readable storage medium. Computer memory may include any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1100 may further include a video display unit 1150, such as a liquid crystal display (LCD), an organic light emitting diode (OLED), a flat panel display, a solid-state display, or a cathode ray tube (CRT). Additionally, the computer system 1100 may include an input device 1160, such as a keyboard/virtual keyboard or touch-sensitive input screen or speech input with speech recognition, and a cursor control device 1170, such as a mouse or touch-sensitive input screen or pad. The computer system 1100 can also include a disk drive unit 1180, a signal generation device 1190, such as a speaker or remote control, and a network interface device 1140.

In an embodiment, as depicted in FIG. 11, the disk drive unit 1180 may include a computer-readable medium 1182 in which one or more sets of software instructions 1184, e.g. software, can be embedded. Sets of software instructions 1184 can be read from the computer-readable medium 1182. Further, the software instructions 1184, when executed by a processor, can be used to perform one or more of the methods and processes as described herein. In an embodiment, the software instructions 1184 may reside completely, or at least partially, within the main memory 1120, the static memory 1130, and/or within the processor 1110 during execution by the computer system 1100.

In an alternative embodiment, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionalities as described herein, and a processor described herein may be used to support a virtual processing environment.

The present disclosure contemplates a computer-readable medium 1182 that includes software instructions 1184 or receives and executes software instructions 1184 responsive to a propagated signal; so that a device connected to a network 1101 can communicate voice, video or data over the network 1101. Further, the software instructions 1184 may be transmitted or received over the network 1101 via the network interface device 1140.

Accordingly, dynamic tissue imagery updating enables presentation of updated pre-operative imagery in a way that reflects how the underlying subject matter has changed since being first generated. In this way, clinicians such as surgeons involved in an interventional medical procedure can view anatomy in a way that reduces confusion and requirements for reorientation during an interventional medical procedure, which in turn improves outcomes of medical interventions.

Although dynamic tissue imagery updating has been described with reference to several exemplary embodiments, it is understood that the words that have been used are words of description and illustration, rather than words of limitation. Changes may be made within the purview of the appended claims, as presently stated and as amended, without departing from the scope and spirit of dynamic tissue imagery updating in its aspects. Although dynamic tissue imagery updating has been described with reference to particular means, materials and embodiments, dynamic tissue imagery updating is not intended to be limited to the particulars disclosed; rather dynamic tissue imagery updating extends to all functionally equivalent structures, methods, and uses such as are within the scope of the appended claims.

For example, while dynamic tissue imagery updating has been described largely in the context of lung surgery, dynamic tissue imagery updating may be applied to any surgery in which deformable tissue is to be tracked. Dynamic tissue imagery updating can be utilized in any procedure involving deformable tissue or organs, and this includes applications such as lung surgery, breast surgery, colorectal surgery, skin tracking or orthopedics.

Although the present specification describes components and functions that may be implemented in particular embodiments with reference to particular standards and protocols, the disclosure is not limited to such standards and protocols. For example, standards such as BLUETOOTH® may represent examples of the state of the art. Such standards are periodically superseded by more efficient equivalents having essentially the same functions. Accordingly, replacement standards and protocols having the same or similar functions are considered equivalents thereof.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A controller for dynamically updating imagery of tissue during an interventional medical procedure, comprising:
a memory that stores instructions; and
a processor that executes the instructions, wherein, when executed by the processor, the instructions cause the controller to implement a process, comprising:
obtaining pre-operative imagery of the tissue in a first modality;
registering the pre-operative imagery of the tissue in the first modality with a set of sensors adhered to the tissue for the interventional medical procedure;
receiving, from the set of sensors, sets of electronic signals for positions of the set of sensors;
computing geometry of the positions of the set of sensors for each set of the sets of electronic signals;
computing movement of the set of sensors based on changes in the geometry of the positions of the set of sensors between sets of electronic signals from the set of sensors; and
updating the pre-operative imagery to updated imagery to reflect changes in the tissue based on the movement of the set of sensors.

2. The controller of claim 1, wherein the process implemented when the processor executes the instructions further comprises:
applying a first algorithm to each set of the sets of electronic signals to compute the movement of the set of sensors, wherein the sets of electronic signals received from the set of sensors comprise position vectors of positions of the set of sensors sent in real-time, and wherein the set of sensors comprise inertial sensors that each include at least one of a gyroscope or an accelerometer.

3. The controller of claim 2, wherein the process implemented when the processor executes the instructions further comprises:
applying a second algorithm to the pre-operative imagery to update the pre-operative imagery to the updated imagery to reflect the changes in the tissue based on the movement of the set of sensors.

4. The controller of claim 1, wherein the process implemented when the processor executes the instructions further comprises:
registering the pre-operative imagery in the first modality with imagery of the tissue in a second modality.

5. The controller of claim 1, wherein the process implemented when the processor executes the instructions further comprises:
optimizing placement of at least one sensor of the set of sensors based on analyzing images of the tissue.

6. The controller of claim 1, wherein the process implemented when the processor executes the instructions further comprises:
calculating initial positions of each sensor of the set of sensors based on camera images that include the set of sensors; and
registering the camera images to the set of sensors.

7. The controller of claim 1, wherein the pre-operative imagery of the tissue in the first modality is registered with the set of sensors before the movement of the set of sensors is computed based on changes in the geometry of the set of sensors between sets of electronic signals from the set of sensors.

8. The controller of claim 1, wherein the process implemented when the processor executes the instructions further comprises:
generating a three-dimensional model of the tissue based on the geometry of the set of sensors with respect to at least one of the pre-operative imagery of the tissue or the updated imagery of the tissue;
updating the three-dimensional model of the tissue based on each of a plurality of sets of the electronic signals from the set of sensors; and
creating an updated virtual rendering of the pre-operative imagery reflecting a current state of the tissue by updating the pre-operative imagery.

9. The controller of claim 1, wherein the process implemented when the processor executes the instructions further comprises:
recording positional information from each of three axes for each sensor of the set of sensors before receiving the sets of electronic signals from the set of sensors.

10. The controller of claim 1, wherein the process implemented when the processor executes the instructions further comprises:
identifying an activity during the interventional medical procedure based on a frequency of oscillatory motion in the movement.

11. An apparatus configured to dynamically update imagery of tissue during an interventional medical procedure, comprising:
a memory that stores instructions and pre-operative imagery of the tissue obtained in a first modality;
a processor that executes the instructions to register the pre-operative imagery of the tissue in the first modality with a set of sensors adhered to the tissue for the interventional medical procedure; and
an input interface via which sets of electronic signals are received, from the set of sensors, for positions of the set of sensors, wherein the processor is configured to compute geometry of the positions of the set of sensors for each set of the sets of electronic signals and to compute movement of the set of sensors based on changes in the geometry of the positions of the set of sensors between sets of electronic signals from the set of sensors,
wherein the apparatus updates the pre-operative imagery to updated imagery that reflects changes in the tissue based on the movement of the set of sensors and controls a display to display the updated imagery for each set of electronic signals from the set of sensors.

12. The apparatus of claim 11, further comprising:
a feedback interface configured to provide haptic feedback based on a determination that the movement exceeds a predetermined threshold.

13. A system for dynamically updating imagery of tissue during an interventional medical procedure, comprising:
a sensor adhered to the tissue and including a power source that powers the sensor, an inertial electronic component that senses movement of the sensor, and a transmitter that transmits electronic signals indicating the movement of the sensor; and
a controller comprising a memory that stores instructions and a processor that executes the instructions, wherein, when executed by the processor, the controller implements a process that includes:
obtaining pre-operative imagery of the tissue in a first modality;
registering the pre-operative imagery of the tissue in the first modality with the sensor;
receiving, from the sensor, electronic signals for movement sensed by the sensor;
computing geometry of the sensor based on the electronic signals; and
updating the pre-operative imagery to reflect changes of the tissue based on the geometry.

14. The system of claim 13, wherein the sensor further includes:
a sterile protective casing that encloses the power source, the inertial electronic component and the transmitter; and
a biocompatible adhesive to attach to the tissue.

15. The system of claim 13, wherein the power source is energized by light or sound received during the interventional medical procedure.

16. The system of claim 13, wherein the sensor is within the tissue.

17. The system of claim 13, wherein the process implemented when the processor executes the instructions further comprises:
applying a first algorithm to the electronic signals to compute the movement of the sensor, wherein the electronic signals received from the sensor comprise position vectors of positions of the sensor sent in real-time, and wherein the sensor comprises an inertial sensor that includes at least one of a gyroscope or an accelerometer.

18. The system of claim 17, wherein the process implemented when the processor executes the instructions further comprises: applying a second algorithm to the pre-operative imagery to update the pre-operative imagery to the updated imagery to reflect the changes in the tissue based on the movement of the sensor.

19. The system of claim 13, wherein the process implemented when the processor executes the instructions further comprises:
registering the pre-operative imagery in the first modality with imagery of the tissue in a second modality.

20. The system of claim 13, wherein the process implemented when the processor executes the instructions further comprises:
optimizing placement of the sensor based on analyzing images of the tissue.
